SEQUENCE LISTING (4 sequences)

SEQ ID NO: 1 - protein, 107 aa, human [sequence omitted]

SEQ ID NO: 2 - protein, 116 aa, human [sequence omitted]

SEQ ID NO: 3 - protein, 214 aa, human
DIQMTQSPSSVSASIGDRVTITCRASQGIDNWLGWYQQKPGKAPKLLIYDASNLDTGVPSRFSGSGSGTYFTLTISSLQAEDFAVYFCQQAKAFPPTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 446 aa, human
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2019/0125864 A1
(43) Pub. Date: May 2, 2019

(54) COMBINATION THERAPY OF RAMUCIRUMAB AND ABEMACICLIB FOR USE IN TREATMENT OF MANTLE CELL LYMPHOMA

(71) Applicant: Eli Lilly and Company, Indianapolis, IN (US)

(72) Inventor: Andrew Edward SCHADE, Fishers, IN (US)

(21) Appl. No.: 16/092,605

(22) PCT Filed: Apr. 7, 2017
(86) PCT No.: PCT/US2017/026536

§ 371 Date: Oct. 10, 2018

Related U.S. Application Data
(60) Provisional application No. 62/322,973, filed on Apr. 15, 2016.

(51) Int. Cl. A61K 39/395; A61K 31/506; A61P 35/00; A61K 9/00

(52) U.S. Cl. CPC A61K 39/3955; A61K 31/506; A61K 2039/545; A61K 9/0019; A61P 35/00

(57) ABSTRACT

The disclosure relates to a combination of an anti-human VEGFR2 antibody ramucirumab (Ramucirumab or I MC-1121B is a fully human IgG1 specific for the VEGF receptor 2), and abemaciclib (a selective CDK4/6 inhibitor LY2835219), or a pharmaceutically acceptable salt thereof, and to methods of using the combination to treat mantle cell lymphoma (Mantle cell lymphoma -MCL- is an uncommon subtype of non-Hodgkin lymphoma).

The present invention relates to a combination of an anti-human VEGFR2 antibody, preferably ramucirumab, and a CDK4 and 6 inhibitor, preferably, abemaciclib, and to methods of using the combination to treat certain disorders, such as mantle cell lymphoma.

Mantle cell lymphoma is a rare, B-cell Non-Hodgkins Lymphoma (NHL) that most often affects men over the age of 60. The disease may be aggressive but it can also behave in a more indolent fashion in some patients. Mantle cell lymphoma comprises about five percent of all NHLs. The disease is called “mantle cell lymphoma” because the tumor cells originally come from the “mantle zone” of the lymph node. Mantle cell lymphoma is usually diagnosed as a late-stage disease that has typically spread to the gastrointestinal tract and bone marrow.

Ramucirumab (CYRAMZA®) is a fully human monoclonal antibody directed against the vascular endothelial growth factor receptor 2 (VEGFR2). Ramucirumab and methods of making and using this compound including for the treatment of neoplastic diseases such as solid and non-solid tumors are disclosed in WO2003/075840. Ramucirumab is approved by the United States Food and Drug Administration as a single agent or in combination with paclitaxel, for treatment of advanced gastric or gastro-esophageal junction adenocarcinoma, with disease progression on or after prior fluoropyrimidine- or platinum-containing chemotherapy; in combination with docetaxel, for treatment of metastatic non-small cell lung cancer with disease progression on or after platinum-based chemotherapy; and in combination with FOLFIRI (irinotecan, folinic acid, and 5-fluorouracil) chemotherapy, for treatment of metastatic colorectal cancer (mCRC) with disease progression on or after prior therapy with bevacizumab, oxaliplatin, and a fluoropyrimidine.

Abemaciclib (LY2835219), [5-(4-ethyl-piperazin-1-ylmethyl)-pyridin-2-yl]-[5-fluoro-4-(7-fluoro-3-isopropyl-2-methyl-3H-benzoimidazol-5-yl)-pyrimidin-2-yl]-amine, is a CDK inhibitor that targets the CDK4 and. CDK6 cell cycle pathway, with antineoplastic activities. Abemaciclib, including salt forms, and methods of making and using this compound including for the treatment of cancer and more preferably for the treatment of mantle cell lymphoma are disclosed in WO2010/075074. Clinical investigations are ongoing for abemaciclib as a single agent in mantle cell lymphoma. Also, in one arm of an ongoing Phase 1b study, patients with non-small cell lung cancer are receiving combination therapy with ramucirumab and abemaciclib.

Abemaciclib has the following structure:

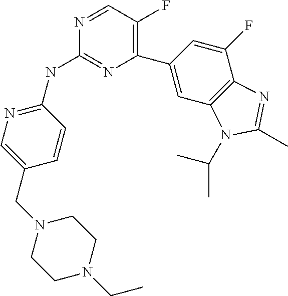 [SMILES: CCN1CCN(Cc2ccc(Nc3ncc(F)c(-c4cc(F)c5nc(C)n(C(C)C)c5c4)n3)nc2)CC1]

Broadly applicable therapies for cancer, in particular for mantle cell lymphoma still remains elusive and there exists a need for more and different therapies that may prove to be effective in treating mantle cell lymphoma. Given the available data and the critical pathways involved in mantle cell lymphoma, the combination of abemaciclib and ramucirumab may provide a new treatment option for patients. Also this may provide a new treatment option for patients who have failed ibrutinib, bortezomib, or lenalidomide.

According to the first aspect of the present invention, there is presented a method of treating mantle cell lymphoma in a patient, comprising administering to the patient an effective amount of an antibody comprising a light chain variable region (LCVR) amino acid sequence of SEQ ID NO: 1 and a heavy chain variable region (HCVR) amino acid sequence of SEQ ID NO: 2, and a compound which is abemaciclib, or a pharmaceutically acceptable salt thereof. Preferably the antibody comprises a light chain amino acid sequence of SEQ ID NO: 3, and a heavy chain amino acid sequence of SEQ ID NO: 4. More preferably the antibody is ramucirumab.

According to another aspect of the present invention, there is presented a kit comprising an antibody comprising a light chain variable region (LCVR) amino acid sequence of SEQ ID NO: 1, and a heavy chain variable region (HCVR) amino acid sequence of SEQ ID NO: 2, and abemaciclib, or a pharmaceutically acceptable salt thereof, for the treatment of mantle cell lymphoma. Preferably the antibody comprises a light chain amino acid sequence of SEQ ID NO: 3, and a heavy chain amino acid sequence of SEQ ID NO: 4. More preferably the antibody is ramucirumab.

According to another aspect of the present invention, there is presented a kit comprising ramucirumab, with one or more pharmaceutically acceptable carriers, diluents, or excipients, and abemaciclib, or a pharmaceutically acceptable salt thereof, with one or more pharmaceutically acceptable carriers, diluents, or excipients, for the treatment of mantle cell lymphoma. Preferably abemaciclib, or the pharmaceutically acceptable salt thereof, is a tablet. Also preferably, abemaciclib, or a pharmaceutically acceptable salt thereof, is a capsule.

According to another aspect of the present invention, there is presented a combination comprising an anti-VEGFR2 antibody and abemaciclib, or a pharmaceutically acceptable salt thereof, for simultaneous, separate, or sequential use in the treatment of mantle cell lymphoma. Preferably the anti-VEGFR2 antibody is ramucirumab.

According to another aspect of the present invention, there is presented an anti-VEGFR2 antibody for use in simultaneous, separate, or sequential combination with abemaciclib,or a pharmaceutically acceptable salt thereof, in the treatment of mantle cell lymphoma. Preferably the anti-VEGFR2 antibody is ramucirumab.

According to another aspect of the present invention, there is presented abemaciclib, or a pharmaceutically acceptable salt thereof, for use in simultaneous, separate, or sequential combination with an anti-VEGFR2 antibody in the treatment of mantle cell lymphoma. Preferably the anti-VEGFR2 antibody is ramucirumab.

The present invention also provides for use of ramucirumab in the manufacture of a medicament for the treatment of mantle cell lymphoma wherein the medicament is to be administered in simulataneous, separate, or sequential combination with abemaciclib, or a pharmaceutically acceptable salt thereof.

The present invention also provides for use of abemaciclib, or a pharmaceutically acceptable salt thereof in the manufacture of a medicament for the treatment of mantle cell lymphoma wherein the medicament is to be administered in simultaneous, separate, or sequential combination with ramucirumab.

For all of the preceding aspects, the following are preferred dosing. Preferably ramucirumab is administered on days 1 and 15 of a 28-day cycle at a dose of 5 mg/kg to 12 mg/kg in separate, or sequential combination with abemaciclib, or a pharmaceutically acceptable salt thereof, wherein abemaciclib, or a pharmaceutically acceptable salt thereof, is administered at a dose of 50 mg to 200 mg twice a day. Preferably abemaciclib or the pharmaceutically salt thereof is administered at a dose of 50 mg to 200 mg twice a day. Also preferably, abemaciclib or the pharmaceutically salt thereof is administered at a dose of 100 mg to 150 mg twice a day. More preferably abemaciclib or the pharmaceutically salt thereof is administered at a dose of 100 mg twice a day. Also more preferably, abemaciclib or the pharmaceutically salt thereof is administered at a dose of 150 mg twice a day. Preferably abemaciclib is administered orally. More preferably abemaciclib is administered by capsule. Also more preferably abemaciclib is administered by tablet. Preferably ramucirumab is administered on days 1 and 15 of a 28-day cycle at a dose of 5 mg/kg to 12 mg/kg. Also preferably, ramucirumab is administered on days 1 and 15 of a 28-day cycle at a dose of 5 mg/kg to 8 mg/kg. More preferably ramucirumab is administered on days 1 to 15 of a 28-day cycle at a dose of 8 mg/kg. More preferably ramucirumab is administered on days 1 to 15 of a 28-day cycle at a dose of 6 mg/kg. More preferably ramucirumab is administered on days 1 to 15 of a 28-day cycle at a dose of 5 mg/kg. Preferably ramucirumab is administered by intravenous infusion. Preferably abemaciclib is administered orally at a dose of 100 mg twice a day and ramucirumab is administered on days 1 and 15 of a 28-day cycle at a dose of 8 mg/kg by intravenous infusion. Preferably abemaciclib is administered orally at a dose of 150 mg twice a day and ramucirumab is administered on days 1 and 15 of a 28-day cycle at a dose of 8 mg/kg by intravenous infusion. Preferably abemaciclib is administered orally at a dose of 100 mg twice a day and ramucirumab is administered on days 1 and 15 of a 28-day cycle at a dose of 6 mg/kg by intravenous infusion. Preferably abemaciclib is administered orally at a dose of 150 mg twice a day and ramucirumab is administered on days 1 and 15 of a 28-day cycle at a dose of 6 mg/kg by intravenous infusion. Preferably abemaciclib is administered orally at a dose of 100 mg twice a day and ramucirumab is administered on days 1 and 15 of a 28-day cycle at a dose of 5 mg/kg by intravenous infusion. Preferably abemaciclib is administered orally at a dose of 150 mg twice a day and ramucirumab is administered on days 1 and 15 of a 28-day cycle at a dose of 5 mg/kg by intravenous infusion.

As used herein, the term “VEGFR2” refers to Vascular Endothelial Growth Factor Receptor 2, which is known in the art. VEGFR2 is also known as KDR.

As used herein, the term “anti-VEGFR2 Ab” refers to an antibody comprising: a light chain variable region (LCVR) whose amino acid sequence is that given in SEQ ID NO: 1, and a heavy chain variable region (HCVR) whose amino acid sequence is that given in SEQ ID NO: 2, wherein the anti-VEGFR2 Ab binds to VEGFR2 with sufficient affinity and specificity. In some embodiments, an anti-VEGFR2 Ab is an antibody comprising: a light chain whose amino acid sequence is that given in SEQ ID NO: 3, and a heavy chain whose amino acid sequence is that given in SEQ ID NO: 4 and that binds to VEGFR2 with sufficient affinity and specificity. In other embodiments of the present invention the anti-VEGFR2 Ab is ramucirumab. The antibody selected will have a sufficiently strong binding affinity for VEGFR2. For example, the antibody will generally bind VEGFR2 with a K_{d} value of between about 100 nM-about 1 pM. Antibody affinities may be determined by a surface plasmon resonance based assay (such as the BIAcore assay is described in PCT Application Publication No. WO2005/012359); enzyme-linked immunosorbent assay (ELISA); and competition assays (e.g. a radiolabeled antigen binding assay (RIM), for example. In one embodiment, Kd is measured by a RIA performed with an anti-VEGFR2 Ab, preferably ramucirumab.

As used herein, the term “ramucirumab” also known as CYRAMZA®, IMC-1121b or CAS registry number 947687-13-0, refers to an anti-VEGFR2 Ab comprising: two light chains, each of whose amino acid sequence is that given in SEQ ID NO: 3, and two heavy chains, each of whose amino acid sequence is that given in SEQ ID NO: 4.

Unless indicated otherwise, the term “antibody” or “Ab” refers to an immunoglobulin molecule comprising two heavy chains (HC) and two light chains (LC) interconnected by disulfide bonds. The amino terminal portion of each chain includes a variable region of about 100 to about 110 amino acids primarily responsible for antigen recognition via the complementarity determining regions (CDRs) contained therein. The carboxy-terminal portion of each chain defines a constant region primarily responsible for effector function.

As used herein, the term “light chain variable region” or “LCVR” refers to a portion of a light chain of an antibody molecule that includes amino acid sequences of CDRs and FRs.

As used herein, the term “heavy chain variable region” “HCVR” refers to a portion of a heavy chain of an antibody molecule that includes amino acid sequences of CDRs and FRs.

As used herein, the term “kit” refers to a package comprising at least two separate containers, wherein a first container contains abemaciclib, or a pharmaceutically acceptable salt thereof, and a second container contains an anti-VEGFR2 Ab. A “kit” may also include instructions to administer all or a portion of the contents of these first and second containers to a cancer patient, preferably a mantle cell lymphoma patient.

As used herein, the terms “treating”, “to treat”, or “treatment” refers to restraining, slowing, stopping, reducing, shrinking, maintaining stable disease, or reversing the progression or severity of an existing symptom, disorder, condition, or disease.

As used herein, the term “patient” refers to a mammal, preferably a human.

As used herein, the terms “cancer” and “cancerous” refer to or describe the physiological condition in patients that is typically characterized by unregulated cell proliferation. Included in this definition are benign and malignant cancers.

As used herein, the term “effective amount” refers to the amount or dose of abemaciclib, or a pharmaceutically acceptable salt thereof, and the amount or dose of an anti-VEGFR2 Ab which provides an effective response in the patient under diagnosis or treatment.

As used herein, the term “effective response” of a patient or a patient's “responsiveness” to treatment with a combination of agents refers to the clinical or therapeutic benefit imparted to a patient upon administration of abemaciclib, or a pharmaceutically acceptable salt thereof and an anti-VEGFR2 Ab.

As used herein, the term “in combination with” refers to the administration of abemaciclib, or a pharmaceutically acceptable salt thereof and an anti-VEGFR2 Ab, preferably ramucirumab, either simultaneously or sequentially in any order, such as for example, at repeated intervals as during a standard course of treatment for a single cycle or more than one cycle, such that one agent can be administered prior to, at the same time, or subsequent to the administration of the other agent, or any combination thereof.

A main advantage of the combination treatments of the invention is the ability of producing marked anti-cancer effects in a patient without causing significant toxicities or adverse events, so that the patient benefits from the combination treatment method overall. The efficacy of the combination treatment of the invention can be measured by various endpoints commonly used in evaluating cancer treatments, including but not limited to, tumor regression, tumor weight or size shrinkage, time to progression, overall survival, progression free survival, overall response rate, duration of response, and quality of life. The therapeutic agents used in the invention may cause inhibition of metastatic spread without shrinkage of the primary tumor, may induce shrinkage of the primary tumor, or may simply exert a tumoristatic effect. Because the invention relates to the use of a combination of unique anti-tumor agents, novel approaches to determining efficacy of any particular combination therapy of the present invention can be optionally employed, including, for example, measurement of plasma or urinary markers of angiogenesis and/or cell cycle activity, tissue-based biomarkers for angiogenesis and/or cell cycle activity, and measurement of response through radiological imaging.

Dosages per day of abemaciclib, or a pharmaceutically acceptable salt thereof normally fall within the range of about 50 mg to 200 mg twice daily, more preferably 100-150 mg twice daily. Most preferably 100 mg twice daily. Also most preferable 150 mg twice daily.

Dosages of ramucirumab per 28-day cycle cycle normally fall within the range of 5 mg/kg to 12 mg/kg given on days 1 and 15, preferably 5 mg/kg to 8 mg/kg given on days 1 and 15, and most preferably 8 mg/kg given on days 1 and 15. Also preferable is 6 mg/kg given on days 1 and 15, Also preferable is 5 mg/kg given on days 1 and 15.

When given in combination with an anti-VEGFR2 Ab, for example, over a 28-day cycle, abemaciclib, or a pharmaceutically acceptable salt thereof, is administered daily within the range of 50 mg to 200 mg twice a day and an anti-VEGFR2 Ab, preferably ramucirumab, is administered on day 1 and 15 within the range of 5 mg/kg to 12 mg/kg.

When given in combination with an anti-VEGFR2 Ab, for example, over a 28-day cycle, abemaciclib, or a pharmaceutically acceptable salt thereof, is administered daily within the range of 100 mg to 150 mg twice a day and an anti-VEGFR2 Ab, preferably ramucirumab, is administered on day 1 and 15 within the range of 5mg/kg to 12 mg/kg.

When given in combination with an anti-VEGFR2 Ab, for example, over a 28-day cycle, abemaciclib, or a pharmaceutically acceptable salt thereof, is administered daily within the range of 100 mg twice a day and an anti-VEGFR2 Ab, preferably ramucirumab, is administered on day 1 and 15 at 8 mg/kg.

When given in combination with an anti-VEGFR2 Ab, for example, over a 28-day cycle, abemaciclib, or a pharmaceutically acceptable salt thereof, is administered daily within the range of 150 mg twice a day and an anti-VEGFR2 Ab, preferably ramucirumab, is administered on day 1 and 15 within the range of 8 mg/kg.

When given in combination with an anti-VEGFR2 Ab, for example, over a 28-day cycle, abemaciclib, or a pharmaceutically acceptable salt thereof, is administered daily within the range of 100 mg twice a day and an anti-VEGFR2 Ab, preferably ramucirumab, is administered on day 1 and 15 at 6 mg/kg,

When given in combination with an anti-VEGFR2 Ab, for example, over a 28-day cycle, abemaciclib, or a pharmaceutically acceptable salt thereof, is administered daily within the range of 150 mg twice a day and an anti-VEGFR2 Ab, preferably ramucirumab, is administered on day 1 and 15 within the range of 6 mg/kg.

When given in combination with an anti-VEGFR2 Ab, for example, over a 28-day cycle, abemaciclib, or a pharmaceutically acceptable salt thereof, is administered daily within the range of 100 mg twice a day and an anti-VEGFR2 Ab, preferably ramucirumab, is administered on day 1 and 15 at 5 mg/kg.

When given in combination with an anti-VEGFR2 Ab, for example, over a 28-day cycle, abemaciclib, or a pharmaceutically acceptable salt thereof, is administered daily within the range of 150 mg twice a day and an anti-VEGFR2 Ab, preferably ramucirumab, is administered on day 1 and 15 within the range of 5 mg/kg.

The free base, abemaciclib, is preferred. However, it will be understood by the skilled reader that abemaciclib can react with any of a number of inorganic and organic acids to form pharmaceutically acceptable acid addition salts. Such pharmaceutically acceptable acid addition salts and common methodology for preparing them are well known in the art. See, e.g., P. Stahl, et al., Handbook of Pharmaceutical Salts: Properties, Selection and Use (VCHA/Wiley-VCH, 2002); L. D. Bighley, et al., Encyclopedia of Pharmaceutical Technology, 453-499 (1995); S. M. Berge, et al., Journal of Pharmaceutical Sciences, 66, 1, (1977). The hydrochloride and mesylate salts are preferred salts. The mesylate salt is an especially preferred salt.

Abemaciclib or pharmaceutically acceptable salts thereof, may be prepared by a variety of procedures known in the art (e.g., see WO2010/075074). Ramucirumab can be made, for example, according to the disclosure in WO2003/075840.

The route of administration may be varied in any way, limited by the physical properties of the drugs and the convenience of the patient and the caregiver. Preferably, an anti-VEGFR2 Ab, preferably ramucirumab, is formulated for parenteral administration, such as intravenous or subcutaneous administration. Preferably, abemaciclib, or pharmaceutically acceptable salt thereof, is formulated for oral or parenteral administration, including intravenous or subcutaneous administration.

Abemaciclib may be formulated into a tablet or capsule. Such pharmaceutical compositions and processes for preparing same are well known in the art. (See, e.g., Remington: The Science and Practice of Pharmacy, L. V. Allen, Editor, 22^{nd} Edition, Pharmaceutical Press, 2012).

### EXAMPLE 1

### IVEF Study

This study evaluates the overall sensitivity of the Mino xenograft model for mantle cell lymphoma (MCL) to growth inhibition by abemaciclib. Accordingly, mice are implanted subcutaneously with human Mino xenografts treated once-daily for 28 days (QD×28) with 25 or 50 mg/kg of abemaciclib monomesylate (“compound”). In addition the response of this model to single-agent DC101, an antibody inhibitor of VEGFR2, is also evaluated as well as the combination of this antibody with abemaciclib. DC101, which is a mouse surrogate of ramucirumab, is evaluated in these studies at a dose of 20 mg/kg given twice-weekly over a period of 4 weeks (BIW×4) whereas mice in the combination group are given this dose of DC101 plus 25 mg/kg of abemaciclib. Tumor volume measurements are taken periodically to determine the effects of the various treatments on tumor growth and periodic body weight measurements are used as a general indicator of tolerability.

Female Fox Chase CB17SCID (Harlan Laboratories) mice are used for these studies. The animals are housed until they reach 18-20 grains in size.

The compound is formulated in 1% hydroxyethyl cellulose (HEC) in 25 mM phosphate buffer (PB) pH=2. The compound is formulated on a weekly basis and stored at 4° C. For the efficacy studies, the compound is administered by oral gavage once-daily for 28 days (PO, QD×28) at doses of 25 or 50 mg/kg using 0.2 ml/dose beginning on day 33 after tumor implantation. The control group is given 1% HEC vehicle according to the same schedule for the compound. The mouse surrogate for ramucirumab known as DC101 is formulated in phosphate buffered saline (PBS) and is given twice-weekly (BIW) for 4 weeks as an intraperitoneal (IP) injection at a dose of 20 mg/kg beginning on day 34. Mice in the combination group are given 25 mg/kg of the compound and 20 mg/kg of DC101 following the schedules outlined above for the monotherapy groups.

The human MCL line Mino is grown in RPMI 1640 medium+20% fetal bovine serum (FBS). Sub-confluent cells are harvested and rinsed twice with Hank's balanced saline solution (HBSS) without serum. For subcutaneous tumors, growth is initiated by subcutaneous injection of 5×10⁶ cells in a 1:1 mixture of HBSS and MATRIGEL® (BD Bioscience, Franklin Lakes, N.J.) in the rear flank of each subject animal. When mean tumors volumes reach approximately 150 mm³ in size, the animals are randomized by tumor size and body weight using a proprietary block randomization allocation tool and placed into their respective treatment groups using 7 animals per group.

Tumor size and body weight are captured using WEB DIRECTOR™. Tumor volume (V) is estimated by using the formula: V=0.536L×W² where L=larger of measured diameter and W=smaller of perpendicular diameter. The tumor volume data is transformed to a log scale to equalize variance across time and treatment groups. The log volume data ae analyzed with a two-way repeated measures analysis of variance by time and treatment using the MIXED procedures in SAS software (Version 9.3). The correlation model for the repeated measures is Spatial Power. Treated groups are compared to the control group at each time point. The MIXED procedure is also used separately for each treatment group to calculate adjusted means and standard errors at each time point. Both analyses accounted for the autocorrelation within each animal and the loss of data that occur when animals with large tumors are removed from the study early. The adjusted means and standard errors are plotted for each treatment group versus time.

Relative changes in tumor volume (% ΔT/C) are calculated using the tumor volume measurements taken on the day (day 53) when the control group has to be sacrificed due to tumor sizes that exceeded the threshold defined by ethical guidelines. The baseline tumor volume is the volume recorded on the day just prior to first day of dosing (baseline day=study day 32). % ΔT/C values are calculated using the formula % ΔT/C=100×ΔT/ΔC, whereby T=mean tumor volume of the compound treated group, ΔT=mean tumor volume of the compound treated group minus the mean tumor volume on the baseline day, C=mean tumor volume of the control (vehicle) group, and ΔC=mean tumor volume of the control group minus the mean tumor volume on the baseline day. If ΔT was <0, then a tumor regression value is calculated instead of % ΔT/C whereby % Regression=100×ΔT/Tᵢₙᵢₜᵢₐₗ such that Tᵢₙᵢₜᵢₐₗ=the grand mean of the tumor volume for all the treatment groups.

Growth inhibition is observed in those instances where the calculated values for % ΔT/C are less than 100% whereby greater inhibition results in smaller % ΔT/C values. Calculated values of % ΔT/C greater than 100% indicate instances where the average tumor volume of the treated group is larger than the average tumor volume of the vehicle control group. Any negative values for % ΔT/C listed in the tables are values for % regression whereby the average tumor volume for the treated group is less than the tumor volume measured on the baseline day (before treatment was initiated).

Progressive disease (PD) is defined as an increase in % ΔT/C relative to baseline of ≥20%; stable disease (SD) is defined by tumor volumes which show any measureable increase in tumor volume relative to baseline which is <20% (0%<SD<20%); a partial response (PR) is defined by the range of tumor volumes which either show no growth relative to baseline (0%) or have reductions in tumor volume of 80% or less (0%≥PR≥−80%); and a complete response (CR) is defined by reductions in tumor volume greater than 80% (<−80%). The disease control rate (DCR) for each treatment group is the sum of the animals in that group which achieved a CR, a PR or SD (DCR=SD+PR+CR). The overall response rate (ORR) for a treatment group is the sum of the animals within that group which attained either a PR or CR (ORR=PR+CR).

Treatment of mice bearing Mino tumors with the compound resulted in a dose-dependent inhibition of tumor growth relative to the vehicle control group. In particular, the % ΔT/C that was observed on the last day of survival for the control group (day 53) was approximately 5% following treatment with 25 mg/kg, whereas mean tumor volume was reduced (regression) by 64% (% ΔT/C=−63.6%) with treatments with 50 mg/kg. For all the groups receiving treatment with the compound, the growth inhibition and/or regression observed was statistically significant (p<0.001) as compared to the vehicle control group. On day 53 100% (7 of 7) of the mice treated with 25 mg/kg of abemaciclib achieved stable disease (SD) and 100% (7 of 7) of the mice treated with 50 mg/kg achieved a partial response (PR).

Growth inhibition is also observed with monotherapy with 20 mg/kg DC101, however not to the extent observed with monotherapy with the compound. In particular the % ΔT/C that was observed on the last day of survival for the control group (day 53) was approximately 26% with only 1 of 6 animals achieving stable disease (SD) or better. The growth of the xenograft tumors of the remaining animals was inhibited by DC101 monotherapy, but all of these mice showed some degree of disease progression (PD). The antitumor efficacy observed on day 53 for the DC101 monotherapy group was statistically significant (p<0.001) as compared to the vehicle control group.

Significant antitumor efficacy was observed with the combination of 20 mg/kg of DC101 plus 25 mg/kg of the compound. Specifically, the % ΔT/C that was observed on the last day of survival for the control group (day 53) was approximately 2% for this group. This response was statistically different from vehicle controls (p<0.001) as well as in comparison to the DC101 (p<0.001) monotherapy group The waterfall plots show for the combination group that 5 out of 7 (71%) mice achieved stable disease (SD) and 2 of 7 (29%) achieved a partial response (PR), This compares favorably to the monotherapy groups wherein stable disease was the best response observed whereby 100% of mice achieved this threshold for abemaciclib monotherapy (at 25 mg/kg) and only 26% for DC101 monotherapy. Although the combination therapy on day 53 showed a trend towards a better response when compared to abemaciclib monotherapy, this response was not quite statistically significant (p=0.053). However the differences between the 2 groups became significant by the end of the treatment period (day 60) (p=0.023).

TABLE 1
Growth inhibition of subcutaneous Mino
xenografts by the Compound and DC101 on day 53

| | Day 53 | | | | | | |
|---|---|---|---|---|---|---|---|
| | | Mean | | | | | |
| | | Tumor | | | Significance (p) | | |
| | | Volume | | % | vs. | vs. | vs. |
| Group | Treatment | (mm³) | SE | ΔT/C | Grp 1 | Grp 2 | Grp 4 |
| 1 | Vehicle | 3326.5 | 395.9 | 100.0 | — | — | — |
| | Control | | | | | | |
| 2 | 25 mg/kg | 317.4 | 37.1 | 5.2 | <0.001 | — | — |
| | Com- | | | | | | |
| | pound | | | | | | |
| 3 | 50 mg/kg | 55.4 | 6.2 | −63.6 | <0.001 | <0.001 | — |
| | Com- | | | | | | |
| | pound | | | | | | |
| 4 | 20 mg/kg | 986.0 | 178.8 | 26.3 | <0.001 | <0.001 | — |
| | DC101 | | | | | | |
| 5 | 50 mg/kg | 203.4 | 22.5 | 1.6 | <0.001 | 0.058 | <0.001 |
| | Com- | | | | | | |
| | pound + | | | | | | |
| | DC101 | | | | | | |

Mean growth inhibition as indicated by relative changes in mean tumor volume (% ΔT/C) was determined for each of the treatment groups by assigning the reference points for measuring inhibition to the last day of survival for the control group (day 53) using the mean tumor volume recorded just prior to first day of dosing as the baseline tumor volume (baseline day = study day 32). Growth inhibition is observed in those instances where the calculated values for % ΔT/C are less than 100% whereby greater inhibition results in smaller % ΔT/C values. Negative values for mean % ΔT/C indicate instances of regression whereby the mean tumor volume on day 53 was smaller than the mean baseline tumor volume (day 32).

TABLE 2
Growth of subcutaneous Mino xenografts on day 60

| | Day 60 | | | | |
|---|---|---|---|---|---|
| | | Mean Tumor | | Significance (p) | |
| | | Volume | | vs. Grp | vs. Grp |
| Group | Treatment | (mm³) | SE | 2 | 4 |
| 2 | 25 mg/kg Compound | 348.6 | 40.8 | — | — |
| 3 | 50 mg/kg Compound | 37.9 | 4.2 | <0.001 | — |
| 4 | 20 mg/kg DC101 | 1900.1 | 350.3 | <0.001 | — |
| 5 | 50 mg/kg Compound + | 204.3 | 22.6 | 0.023 | <0.001 |
| | DC101 | | | | |

The tumor volumes measured on the last day of dosing (day 60) are presented in the table. % ΔT/C values could not be calculated on day 60 since none of the animals in the control group survived beyond day 53.

### EXAMPLE 2

### An Open-Label, Phase 1a/1b Study of Ramucirumab in Combination with Other Targeted Agents in Advanced Cancers

### Study Design

The study is a multicenter, nonrandomized, open-label Phase 1a/1b study evaluating the safety and efficacy of ramucirumab in combination with abemaciclib for the treatment of mantle cell lymphoma. Phase 1a will consist of a dose-limiting toxicity (DLT) observation period. Phase 1b will consist of an expansion period. This study is designed to investigate ramucirumab in combination with abemaciclib which was chosen based on scientific rationale, prior clinical experience with other targeted agents, and experience with ramucirumab, to treat patients with various types and stages of cancer. The primary analysis will be conducted at the earlier of the following time points:
1. approximately 1 year after the last patient in the study receives his or her first dose of study treatment in Phase 1b; or
2. after all patients in the study have discontinued all study treatment.

### Study Objectives and Endpoints

The primary objective of this study is to assess the safety and tolerability of ramucirumab plus abemaciclib in specific cancer indications, specifically mantle cell lymphoma.

The secondary objectives of this study are 1) to assess the pharmacokinetics of ramucirumab and abemaciclib when co-administered, and 2) to document the preliminary antitumor activity observed with ramucirumab in combination with abemaciclib for example,
Proportion of Participants Who Exhibit Complete Response (CR) or Partial Response (PR) [Overall Response Rate (ORR)]
Progression Free Survival (PFS).

The exploratory objective of this study is to assess the relationship between biomarkers associated with treatment, treatment mechanism of action, cancer, and immune response to clinical outcome.

The endpoints are indentifying dose limiting toxicities (DLTs)/DLT toxicity, safety monitoring (including adverse events, treatment-emergent adverse events, serious adverse events, and deaths), minimum serum/plasma concentration of ramucirumab and abemaciclib, ORR per lymphoma criteria (Barrington et al 2014; Cheson et al 2014), progression free survival, and optionally biomarker research (on genetic, molecular, and circulating factors from whole blood and tumor tissue samples) unless precluded by local regulations.

### Treatment Plan

All patients enrolled in this study will receive ramucirumab intravenously administered in combination with orally administered abemaciclib during Phase 1a (DLT Observation Period) and Phase 1b (Expansion Period). Phase 1a will last for one treatment cycle. Patients who complete Phase 1a without a DLT will continue treatment until a criteria for discontinuation is met.

Ramucirumab (8 mg/kg administered IV on Day 1 and Day 15 every 28 days) and abemaciclib (100 mg or 150 mg administered orally every 12 hours) are administered in a 28-day treatment cycle during Phase 1a (DLT observation period) of the study. Doses of ramucirumab and/or abemaciclib may be delayed, omitted, or reduced if a patient experiences an adverse event described below or a DLT-equivalent toxicity (defined as a DLT occurring after the DLT observation period) using CTCAE Version 4.0 (NCI 2009) to assign adverse event terms and severity grades. After 1 treatment cycle of 28 days and completion of an interim safety analysis, continuation of ramucirumab and abemaciclib at the above doses may resume for Phase 1b (the expansion period) until a criterion for discontinuation is met.

The following criteria applies to maintaining a control on the enrolled population for the study but should not be construed to limit the potential population that is intended under this patent application.

Initially, 3 patients are enrolled in Phase 1a. Additional patients may be enrolled based on the following criteria:
If none of the initial 3 patients treated at a given dose level develops a DLT, Phase 1b will start.

If 1 of the initial 3 patients treated at a given dose level develops a DLT, 3 additional patients will be enrolled at that dose level.
If ≤1 of the 6 patients treated at a given dose level develops a DLT, Phase 1b will start.
If ≥2 patients treated at a given dose level develop a DLT, enrollment in that study will stop, and an alternative dose level may be considered.

In Phase 1b, 15 additional patients are enrolled.
Patients are eligible to be included if they meet all of the following criteria: Inclusion Critera:

[1] have a diagnosis of the type of cancer listed below and meet the following requirements:
pathologically confirmed mantle cell lymphoma, with (a) measurable nodal disease on positron emission tomography-computed tomography (PET-CT)cross-sectional imaging that is ≥1.5 cm in longest diameter or (b) measureable extra-nodal disease that is ≥1.0 cm and measureable in 2 perpendicular dimensions per CT per Lugano classification (Barrington et al. 2014; Cheson et al. 2014). Prior to enrollment, pathology must be reviewed and confirmed at the investigational site where the patient is entered.
have mantle cell lymphoma that relapsed after or is refractory to (a) first-line combination chemotherapy with or without stem cell transplant and (b) at least 1 other locally available therapy.
provide a newly obtained tumor tissue sample. Tumor tissue biopsies maybe taken by surgical resection, core needle biopsy, or fine needle biopsy.
have not received previous systemic therapy (including investigational agents) targeting programmed cell death protein 1 (PD-1)/PD-1 ligand (PDL-1) or PD-1/PDL-2 signaling pathways. Prior therapy with other immune checkpoint inhibitors, including but not limited to, anti-CD137 antibody or anticytotoxic T-lymphocyte-associated antigen-4 antibody, is not permitted.
have adequate organ function.
are, in the judgment of the investigator, appropriate candidates for experimental therapy after available standard therapies have failed to provide clinical benefit.
have discontinued all previous treatments for cancer and recovered from the acute effects of therapy, other than less than or equal to Grade 2 neuropathy or nonserious and nonlife-threatening toxicities such as alopecia, altered taste, and nail changes.
have a performance status of 0 or 1 on the Eastern Cooperative Oncology Group scale.
men and women must agree to the use an effective method of contraception during the study and for at least 3 months post last dose of study drug administration.
   Women of child-bearing potential must have negative serum and urine pregnancy tests at screening and during each treatment cycle, respectively.

### Exclusion Criteria:

have a serious illness or medical condition including, but not limited to, the following: received an autologous stem cell transplant within 75 days prior to the initial dose of study drug; received an allogeneic stem cell transplant; active or uncontrolled clinically serious infection, including chronic viral hepatitis.
have prior or concurrent malignancies, inclusive of hematologic, primary brain tumor, sarcoma, and other solid tumors, unless in complete remission with no therapy for a minimum of 5 years.
have active gastrointestinal (GI) disease characterized by inflammatory bowel disease, malabsorption syndrome, or frequent Grade 2 or more diarrhea.
are pregnant or breastfeeding.
have previously documented brain metastases, leptomeningeal disease, or uncontrolled spinal cord compression.
have experienced any of the following: a major surgical procedure, significant traumatic injury, non-healing wound, peptic ulcer, or bone fracture less than or equal to 28 days prior to enrollment, or placement of a subcutaneous venous access device less than or equal to 7 days prior to the first dose of study treatment unless the procedure is of low risk of bleeding in the judgment of the investigator.
have an elective or a planned major surgery during the course of the trial.

have a known allergy or hypersensitivity reaction to any of the treatment components.
have uncontrolled hypertension.
have experienced any arterial thromboembolic event within 6 months prior to enrollment.
have experienced any Grade 3 or 4 venous thromboembolic event that is considered by the investigator to be life threatening or that is symptomatic and not adequately treated by anticoagulation therapy, within 6 months prior to enrollment.
have a history of GI perforation and/or fistulae within 6 months prior to enrollment.
have experienced any bleeding episode considered life threatening, or any Grade 3 or 4 GI/variceal bleeding episode in the 3 months prior to enrollment requiring transfusion or endoscopic or operative intervention.
have congestive heart failure or poorly controlled cardiac arrhythmia per New York Heart Association Class II-IV heart disease.

Treatment with the combination may continue for up to 1 year (12 cycles). Patients receiving clinical benefit may continue treatment during continued access. Doses of ramucirumab and/or the abemaciclib may be delayed, omitted, or reduced if the patient experiences an adverse event or a DLT-equivalent toxicity. In case of difficulty in assigning relatedness to one study drug or the other, the doses of both study drugs may be delayed, reduced, or omitted. Ramucirumab dosing may be delayed for up to 28 days, and abemaciclib dosing may be omitted for up to 14 days. ‘Treatment of either or both drug may be resumed at the dose prior to an adverse event or DLT-equivalent toxicity, or may be continued at a reduced dose.

### Dose-Limiting Toxicities

Toxicity is considered dose-limiting if it is deemed at least possible related to either or both study drugs. A patient is considered evaluable for DLTs if he or she (1) receives at least 70% of the dose of the oral drug and completes the DLT observation period or (2) discontinues because of a DLT. Dose-limiting toxicities (DLTs) are defined as any of the following adverse events: Grade 4 thrombocytopenia (unless recovered in 24 hours and in the absence of bleeding) or Grade 3 thrombocytopenia complicated with Grade≥2 bleeding, Grade 4 hematologic toxicity persisting >5 days, Grade≥3 febrile neutropenia, Grade 3 nonhematologic toxicity that occurs despite maximal supportive medical management, and any other clinically significant toxicity deemed to be dose limiting, such as Grade 2 seizures or severe tremors. Exceptions may be made for: alopecia, nausea, vomiting, anorexia, diarrhea, or constipation that can be appropriately controlled and does not persist for >72 hours with treatment, asymptomatic electrolyte disturbance that can be treated with oral substitution therapy or by intravenous infusions requiring <24-hour hospitalization, and transient (≤5 days) Grade 3 elevations of hepatic transaminases ALT and/or AST without evidence of other hepatic injury in the setting of preexisting hepatic metastasis.

### Ramucirumab Administration

Prior to each infusion of ramucirumab, premedicate all patients with an oral or intravenous histamine H1 antagonist, such as diphenhydramine hydrochloride. Additional premedication may be provided at the investigator's discretion. Ramucirumab infusions should be delivered in approximately 60 minutes. The infusion rate should not exceed 25 mg/min. Infusions >60 minutes are permitted in the following situations:

if needed in order to maintain an infusion rate≤25 mg/min, or

if the patient previously experienced a ramucirumab IRR.

The actual dose of ramucirumab to be administered will be determined by measuring the patient's weight in kilograms at the beginning of each cycle. If the patient's weight fluctuates by more than ±10% from the weight used to calculate the prior dose, the dose MUST be recalculated. Recalculation of the ramucirumab dose for weight fluctuation of <10% is permitted, but not required.

Discontinuation from Study Treatment

Patients will be discontinued from all study treatment (Phase 1a and Phase 1b) in the circumstances listed below. The reason for discontinuation and the date of discontinuation will be collected for all patients.
the patient is enrolled in any other clinical trial involving an investigational product or any other type of medical research judged not to be scientifically or medically compatible with this study;
the patient has progressive disease;
the patient becomes pregnant during the study;
the patient is significantly noncompliant with study procedures and/or treatment,
the patient, for any reason, requires treatment with another therapeutic agent that has been demonstrated to be effective for treatment of the study indication; discontinuation from study treatment will occur prior to introduction of the new agent;
the investigator decides that the patient should be discontinued from study treatment;
the patient requests to be discontinued from study treatment;
the patient's designee (for example, a parent, legal guardian, or caregiver) requests that the patient be discontinued from study treatment.

### Study Assessments

In order to characterize the preliminary efficacy signal observed with ramucirumab in combination with abemaciclib treatment, the ORR (Overall Response Rate) per lymphoma criteria, 5-point Lugano, (Barrington et al. 2014; Cheson et al. 2014) and PFS (Progression-Free Survival) will be analyzed. For patients with fluorodeoxyglucose-avid non-Hodgkin's lymphoma subtypes, PET-CT scans are required. The method of assessment used at baseline must be used consistently throughout the study. Radiological scan of the thorax, abdomen, and pelvis is required. The measures used to assess safety and efficacy in this study are consistent with those used in most conventional oncology trials.

Baseline imaging and measurement are defined by 5-point Lugano criteria. Subsequent radiological imaging is performed according to the 5-point Lugano criteria every 6 weeks (±7 days) for the first 6 months after enrollment and every 9 weeks (±7 days) thereafter until radiographic disease progression, death, or study completion, whichever occurs first. Assessments are performed as scheduled even if study treatment is delayed or omitted, except when deemed not feasible due to the patient's clinical status.

Post—treatment assessment is based on a short and long term follow-up schedule and includes for patients who discontinue study treatment without objectively measured PD (Progressive Disease), a tumor assessment and imaging every 9 to 12 weeks depending on the standard of care using the 5-point Lugano criteria.

Short-term follow-up begins the day after the patient and the investigator agree that the patient will no longer continue study treatment and lasts approximately 30 days (±7 days). No follow-up procedures will be performed for a patient who withdraws informed consent unless he or she has explicitly provided permission and consent.

Long-term follow-up begins the day after short-term follow-up is completed and continues until the patient's death or overall study completion. Follow-up should be attempted at regularly scheduled intervals (every 90 days [±7 days]). This follow-up might be a phone call to the patient, her/his family, or local doctor.

In order to characterize the preliminary efficacy signal, tumor response rates (CR (Complete Response), PR (Partial Response), SD (Stable Disease), ORR) will be provided along with corresponding confidence intervals.

Time-to-event variables, such as time to response, duration of response, and progression-free survival, will be estimated by Kaplan-Meier (1958) methodology. Efficacy will include patients enrolled in Phase 1b as well as patients treated at the recommended dose in Phase 1a. Individual changes in tumor burden over time will be determined.

The primary analysis will be conducted separately at the earlier of the following time points:
1. approximately 1 year after the last patient in the study receives his or her first dose of study treatment in Phase 1b; and
2. after all patients on the study arm have discontinued from all study treatment.
Barrington S F, Mikhaeel N G, Kostakoglu L, Meignan M, Hutchings M, Müeller S P, Schwartz L H, Zucca E, Fisher R I, Trotman J, Hoekstra O S, Hicks R J, O'Doherty M J, Hustinx R, Biggi A, Cheson B D. Role of imaging in the staging and response assessment of lymphoma: consensus of the International Conference on Malignant Lymphomas Imaging Working Group. J Chin Oncol. 2014; 32(27):3048-3058.
Cheson B D, Fisher R I, Barrington S F, Cavalli F, Schwartz L H, Zucca E, Lister T A. Recommendations for initial evaluation, staging, and response assessment of Hodgkin and non-Hodgkin lymphoma: the Lugano classification. J Clin Oncol. 2014; 32(27):3059-3068.

### End of Study

End of the study is the date of the last visit or last scheduled procedure for the last patient.

SEQUENCE LISTING

SEQ ID NO: 1
DIQMTQSPSSVSASIGDRVTITCRASQGIDNWLGWYQQKPGKAPKLLIYD
ASNLDTGVPSRFSGSGSGTYFTLTISSLQAEDFAVYFCQQAKAFPPTFGG
GTKVDIK
SEQ ID NO: 2
EVQLVQSGGGLVKPGGSLRLSCAASGFTFSSYSMNWVRQAPGKGLEWVSS
ISSSSSYIYYADSVKGRFTISRDNAKNSLYLQMNSLRAEDTAVYYCARVT
DAFDIWGQGTMVTVSS
SEQ ID NO: 3
DIQMTQSPSSVSASIGDRVTITCRASQGIDNWLGWYQQKPGKAPKLLIYD
ASNLDTGVPSRFSGSGSGTYFTLTISSLQAEDFAVYFCQQAKAFPPTFGG
GTKVDIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ ID NO: 4
EVQLVQSGGGLVKPGGSLRLSCAASGFTFSSYSMNWVRQAPGKGLEWVSS
ISSSSSYIYYADSVKGRFTISRDNAKNSLYLQMNSLRAEDTAVYYCARVT
DAFDIWGQGTMVTVSSASTKGPSVLPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYNSTYR
VVSLVTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK

1. A method of treating mantle cell lymphoma in a patient, comprising administering to the patient an effective amount of an antibody comprising a light chain variable region (LCVR) amino acid sequence of SEQ ID NO: 1 and a heavy chain variable region (HCVR) amino acid sequence of SEQ ID NO: 2, and a compound which is abemaciclib, or a pharmaceutically acceptable salt thereof.

2. The method of claim 1 wherein the antibody comprises a light chain amino acid sequence of SEQ ID NO: 3, and a heavy chain amino acid sequence of SEQ ID NO: 4.

3. The method of claim 2, wherein the antibody is ramucirumab.

4. The method of claim 3, wherein the compound or salt thereof is administered at a dose of 50 mg to 200 mg twice a day.

5. The method of claim 3, wherein the compound or salt thereof is administered at a dose of 100 mg to 150 mg twice a day.

6. The method of claim 3, wherein the compound or salt thereof is administered at a dose of 100 mg twice a day.

7. The method of claim 3, wherein the compound or salt thereof is administered at a dose of 150 mg twice a day.

8. The method of claim 7 wherein ramucirumab is administered on days 1 and 15 of a 28-day cycle at a dose of 5 mg/kg to 12 mg/kg.

9. The method of claim 7 wherein ramucirumab is administered on days 1 and 15 of a 28-day cycle at a dose of 5 mg/kg to 8 mg/kg.

10. The method of claim 7 wherein ramucirumab is administered on days 1 and 15 of a 28-day cycle at a dose of 8 mg/kg.

11. The method of claim 7 wherein ramucirumab is administered on days 1 and 15 of a 28-day cycle at a dose of 6 mg/kg.

12. The method of claim 7 wherein ramucirumab is administered on days 1 and 15 of a 28-day cycle at a dose of 5 mg/kg.

13-31. (canceled)

32. The method of claim 3 wherein ramucirumab is administered on days 1 and 15 of a 28-day cycle at a dose of 5 mg/kg to 12 mg/kg.

33. The method of claim 3 wherein ramucirumab is administered on days 1 and 15 of a 28-day cycle at a dose of 5 mg/kg to 8 mg/kg.

34. The method of claim 3 wherein ramucirumab is administered on days 1 and 15 of a 28-day cycle at a dose of 8 mg/kg.

35. The method of claim 3 wherein ramucirumab is administered on days 1 and 15 of a 28-day cycle at a dose of 6 mg/kg.

36. The method of claim 7 wherein ramucirumab is administered on days 1 and 15 of a 28-day cycle at a dose of 5 mg/kg.

37. The method of claim 4 wherein ramucirumab is administered on days 1 and 15 of a 28-day cycle at a dose of 8 mg/kg.

38. The method of claim 4 wherein ramucirumab is administered on days 1 and 15 of a 28-day cycle at a dose of 6 mg/kg.

39. The method of claim 4 wherein ramucirumab is administered on days 1 and 15 of a 28-day cycle at a dose of 5 mg/kg.
